(19) United States
(12) Patent Application Publication
(10) Pub. No.: US 2012/0172750 A1
(43) Pub. Date: Jul. 5, 2012

(54) ACCESS CATHETER SYSTEM HAVING A PRECISELY POSTIONABLE NEEDLE TIP

(75) Inventors: Douglas Sawyer, Nashville, TN (US); Michael Barnett, Franklin, TN (US)

(73) Assignee: Vanderbilt University, Nashville, TN (US)

(21) Appl. No.: 12/983,132

(22) Filed: Dec. 31, 2010

(51) Int. Cl. A61B 5/03

(52) U.S. Cl. 600/561

(57) ABSTRACT

An access catheter system, comprising a needle assembly comprising a needle having an elongate lumen configured to be substantially filled with a fluid. The system further comprises a fluid monitor configured to monitor a physical characteristic of fluid within the lumen, and configured to sense a change in the physical characteristic. An indicator provides a indication of the sensed change in the physical characteristic.

### BACKGROUND

1. Field of the Invention

The present invention relates generally to access catheters, and more particularly, to an access catheter system having a precisely positionable needle tip.

2. Related Art

Many surgical and other therapeutic procedures require the precise positioning of the tip of a needle at a specific, relatively small space in a patient. For example, a human patient's body is comprised of layers of tissue that are separated by minimal spaces or layers of fluid, and certain therapeutic procedures require the delivery of drugs between tissue layers. In another example, stimulator electrodes of a pacemaker may need to be located between layers of tissue proximate the interior of a human heart. Still further by example, fluid may need to be drained from such areas between layers of tissue.

Accurately and harmlessly positioning a tip (also referred to herein as a distal end) of a catheter at such spaces is often problematic and may be difficult due to the close proximity of the layers to one another (often separated by less than 1 mm). In particular, it is often difficult to avoid traversing the tip of the needle from one layer of tissue (e.g., an outer layer of tissue that is penetrated with the tip) into another layer of tissue (e.g., an inner layer of tissue that is not desired to be penetrated with the tip).

### SUMMARY

In accordance with one aspect of the present invention, an access catheter system, is provided. The system comprises a needle assembly comprising a needle having an elongate lumen configured to be substantially filled with a fluid; a fluid monitor configured to monitor a physical characteristic of fluid within the lumen, and configured to sense a change in the physical characteristic; and an indicator configured to provide a indication of the sensed change in the physical characteristic.

In accordance with another aspect of the present invention, an operational method of an access catheter including a needle assembly comprising a needle having an elongate lumen configured to be substantially filled with a fluid. The method comprises: monitoring a physical characteristic of the fluid located in the needle lumen; sensing a change in the monitored physical characteristic; and providing an indication of the sensed change.

In accordance with a still other aspect of the present invention, a method to position a distal end of a needle having a fluid-filled lumen at a space and/or a potential space inside a organism adjacent a first side of a first layer of tissue of the organism is provided. The method comprises: inserting the needle into the first layer of tissue through a second side of the first layer of tissue opposite the first side; advancing the needle through the first layer of tissue in a direction from the second side towards the first side; monitoring a physical characteristic of the fluid located in the needle lumen while advancing the needle; and ceasing advancement of the needle when the monitored physical characteristic of the fluid located in the needle lumen indicates that the distal end of the needle has entered the space and/or the potential space.

### BRIEF DESCRIPTION OF THE DRAWINGS

Embodiments of the present invention are described herein with reference to the accompanying drawings, in which:

FIG. 1A is a functional diagram of an access catheter system, in accordance with embodiments of the present invention;

FIG. 1B is a functional diagram of an access catheter system, in accordance with embodiments of the present invention;

FIG. 1C is a functional diagram of an access catheter system, in accordance with embodiments of the present invention;

FIG. 2A is a cross-sectional diagram of an access catheter system, in accordance with embodiments of the present invention;

FIG. 2B is a cross-sectional diagram of an access catheter system, in accordance with embodiments of the present invention;

FIG. 2C is a cross-sectional diagram of an access catheter system, in accordance with embodiments of the present invention;

FIG. 2D is a cross-sectional diagram of an access catheter system, in accordance with embodiments of the present invention;

FIG. 3 is a cross-sectional diagram of an access catheter system, in accordance with embodiments of the present invention;

FIG. 4 is a cross-sectional diagram of an access catheter system, in accordance with embodiments of the present invention;

FIG. 5 is a cross-sectional diagram of an access catheter system, in accordance with embodiments of the present invention;

FIG. 6 is a flow chart detailing steps of a method utilizing an access catheter system according to an exemplary embodiment of the present invention;

FIG. 7 is a flow chart detailing exemplary sub-steps of one of the steps of the method of FIG. 6;

FIG. 8 is a flow chart detailing steps of an alternate method utilizing an access catheter system according to an exemplary embodiment of the present invention;

FIG. 9 is a graphical illustration of the pressure of a fluid in a lumen of an access catheter in accordance with an embodiment of the present invention;

FIG. 10 is another flow chart detailing steps of another method utilizing an access catheter system according to an exemplary embodiment of the present invention; and

FIG. 11 is another flow chart detailing steps of another method utilizing an access catheter system according to an exemplary embodiment of the present invention.

### DETAILED DESCRIPTION

Aspects of the present invention are generally directed to an access catheter system that enables a surgeon or other user to precisely position a needle tip within a patient. Specifically, embodiments of the access catheter system are configured to provide a user with an indication when the needle tip is a desired location. More specifically, the access catheter system includes lumen within the needle that is configured to be substantially filled with a fluid, a fluid monitor and an indicator. As the tip of the needle is advanced in the patient, the monitor senses changes in a physical characteristic (pressure, volume, flow of fluid into or out of the lumen, etc.) of the fluid within the lumen. The indicator provides a notification to the user when a change in the physical characteristic is sensed. Methods for using the access catheter of the present invention are also described in further detail below.

The devices, systems and methods disclosed herein may be utilized in a variety of medical procedures in which relatively precise, reliable and/or quick positioning a tip of a needle at a desired location within a patient is desired at minimal risk to adjacent tissue. For example, such medical procedures may include accessing a space adjacent an internal sac of a patient, such as, for example: a space in the interior of the amniotic sac (to access amniotic fluid and/or the fetus) and/or a space between the chorion and the amnion; a space in the pleura cavity and/or a space in between the parietal and visceral layers of the pleura; a space inside the peritoneum and/or a space in between the parietal peritoneum and the viceral peritoneum; a space inside a blood vessel (e.g., artery, vein, capillaries); and a space inside the pericardium and/or a space between the fibrous pericardium and the serous pericardium.

Still further by way of example, the devices, systems and methods disclosed herein may be utilized to execute pericardial space infusion or drainage, pleural space infusion or drainage, amniocentesis, peritoneal space infusion or drainage, joint space infusion or drainage, intraocular fluid space infusion or drainage, epicardial pacemaker insertion, subdural and/or epidural infusion, pericardioscopy and intracranial pressure monitoring.

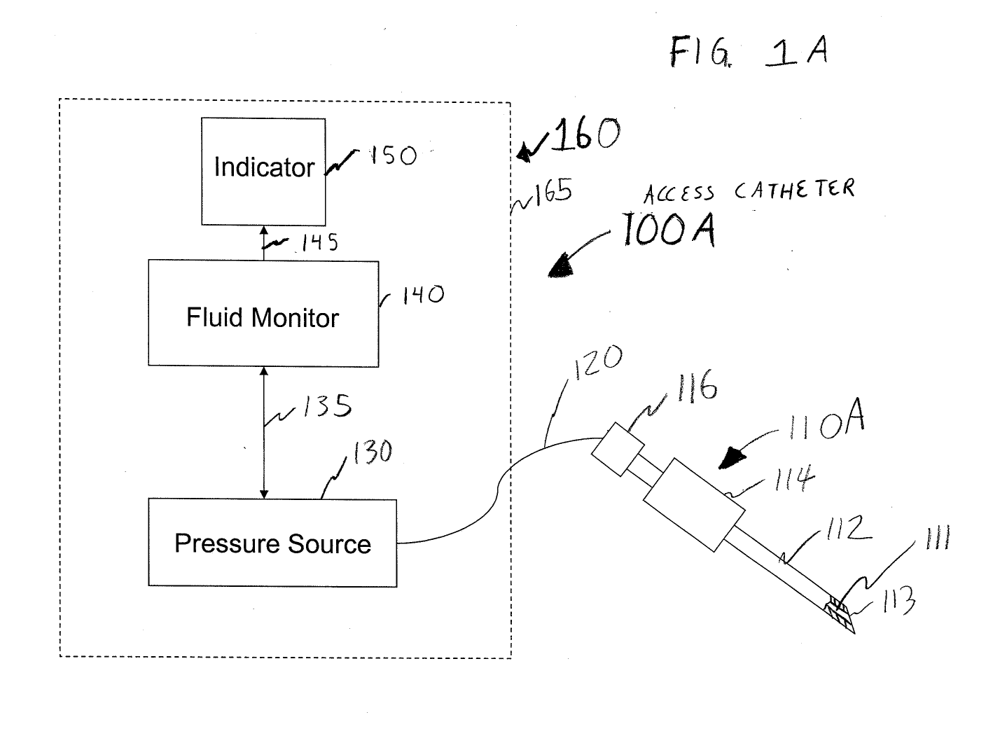

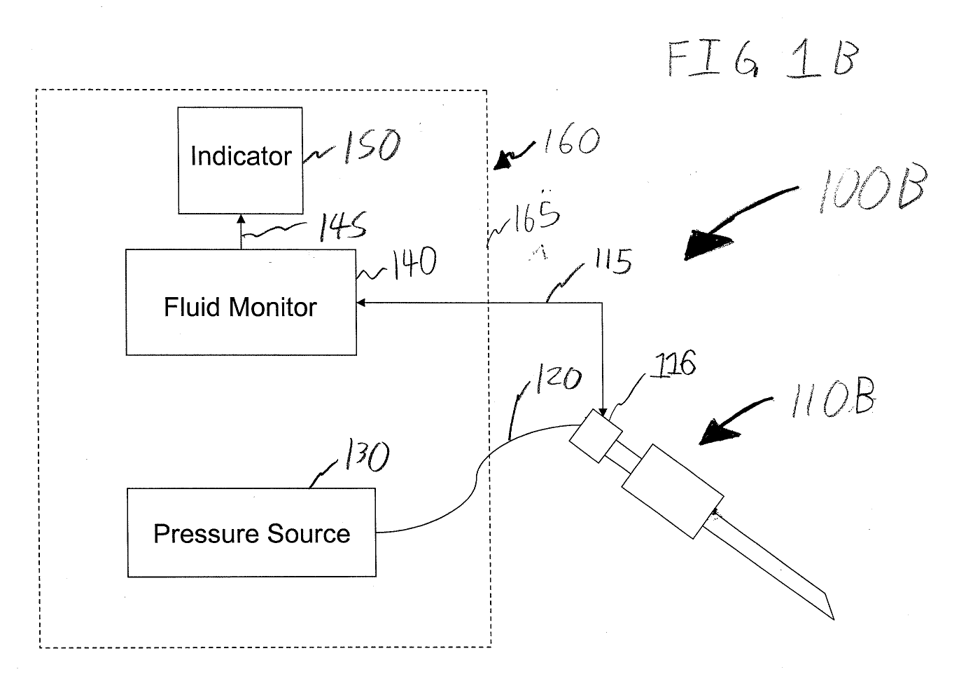

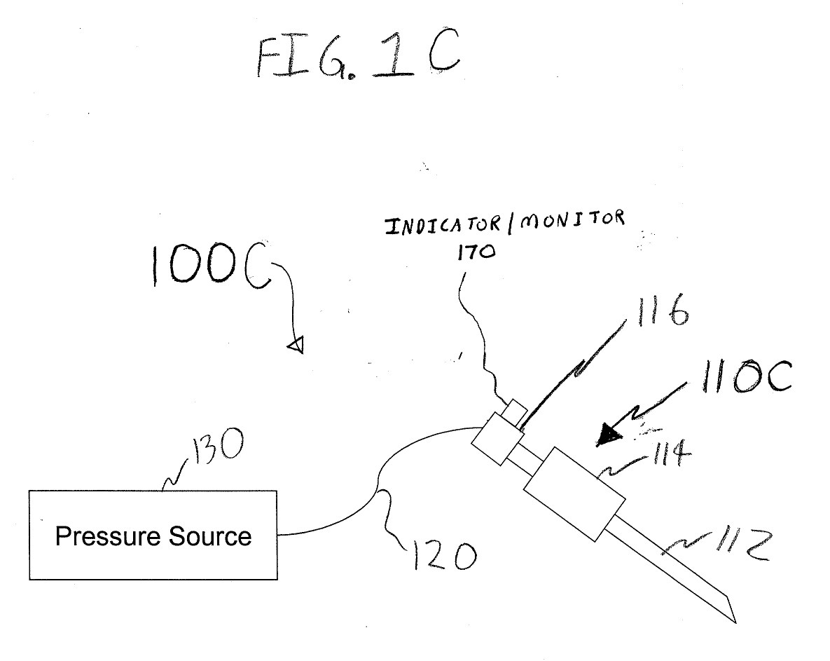

Various embodiments of the present invention will now be described with respect to the drawings. In this regard, FIG. 1A, there is an access catheter 100A according to an exemplary embodiment of the present invention. The access catheter 100A includes catheter needle assembly 110A that is fluidically coupled via fluid supply line 120 to pressure source 130. Pressure source 130 pressurizes a fluid located in the catheter needle assembly 110A, or, more specifically, a fluid in lumen 111 of a transcutaneous needle 112. As described in greater detail below, in the absence of substantial resistance by tissue, the pressurization of the fluid in the catheter needle assembly 110 drives the fluid out of the end of needle 112 at the tip 113.

Needle 112 is configured to penetrate tissue. In the embodiments of FIG. 1A, tip 113 of is beveled, and in an exemplary embodiment, the angle of beveling is shallow. Surrounding needle 112 or otherwise attached to needle 112 is a handle 114. The handle 114 facilitates manipulation of the catheter needle assembly 110A during the invasive medical exploratory procedure, and may be spatially adjustable along the length of needle 112 and/or about the longitudinal axis of needle 112. The catheter needle assembly 110A also optionally includes a fluid coupling 116 that permits the fluidic coupling of the fluid supply line 120 to lumen 111 of needle 112.

Pressure source 130 may be a pump or a volume of fluid under pressure, etc., that pressurizes fluid in lumen 111 of needle 112. In an exemplary embodiment, the pressure source is a reciprocating pump that pressurizes and depressurizes (at least relative to the pressure resulting from the pressurization), in a pulsating manner, the fluid in lumen 111 of needle 112 such that the flow of fluid out of needle 112 at tip 113 is minimized. In an exemplary embodiment, the pressurization and depressurization of the fluid in lumen has the cumulative result that a relatively low amount of fluid is driven from needle 112 at tip 113, because at least some of the fluid driven out of needle 112 during the pressurization phase is subsequently pulled back into needle during the depressurization phase.

In an exemplary embodiment, the pressurization and depressurization of the fluid in lumen 111 of needle 112 provides a “water hammer” effect, or a “micro-water hammer” effect at tip 113 of needle 112. As would be appreciated, use of the phrase “water hammer” is not intended to limit embodiments of the present invention to the use of water as the fluid in lumen 111. Rather, embodiments of the present invention may utilize a number of other fluids, such as saline, to achieve the water hammer effect at tip 113 of needle 112. In an embodiment of the present invention, water hammer is achieved by repeatedly (i) increasing pressure of the fluid in the lumen of needle to a first pressure greater than a second pressure (which may correspond to pressurization of the fluid in the lumen 111, detailed above), and (ii) permitting the pressure of the fluid in the needle lumen to decrease from the first pressure to or at least towards the second pressure.

In an alternative embodiment of the present invention, pressure source 130 may be a volume of fluid under a static pressure. In these embodiments, the static pressure is transferred via fluid supply line 120 to lumen 111 to pressurize the fluid therein.

The embodiments of FIG. 1A schematically depict pressure source 130 in communication with a fluid monitor 140 via a communication line 135. In one embodiment, communication line 135 may be electrical leads electrically in the case of electrical communication, and may be in the form of piping and/or flexible tubing in the case of fluidic communication. Fluid monitor 140 monitors a physical characteristic of the fluid that is located in lumen 111 of needle 112. By way of example and not by way of limitation, the monitored physical characteristic of the fluid may be a pressure of the fluid and/or a change in pressure of the fluid. Other physical characteristics of the fluid that may be monitored may include a flow rate of the fluid in lumen 111 and/or a change in the flow rate of the fluid in lumen 111, etc., as will be described in greater detail below. In some embodiments, any characteristic of the fluid that is indicative of fluid flow through lumen 111 out of tip 113 of needle 112 and/or a change in fluid flow out of tip 113 may be monitored by fluid monitor 140.

As noted above, fluid monitor 140 senses changes in the physical characteristic(s) of the fluid located in lumen of needle 112. In certain embodiments, the physical characteristic(s) of the fluid in lumen 111 may be directly or monitored, while in other embodiments the characteristic(s) are indirectly monitored via pressure source 130. That is, because pressure source 130 is in fluid communication with lumen 111 via fluid supply line 120, monitoring the physical characteristic(s) of the fluid located in, or proximate to pressure source 130, permits the physical characteristic of the fluid located in the lumen 111 to be monitored. The physical characteristic of the fluid may be monitored in this manner by, for example, utilizing a transducer located in/at pressure source 130 and/or fluid supply line 120. In alternative or additional embodiments, the physical characteristic of the fluid located in pressure source 130 may be monitored indirectly by monitoring an ancillary feature of the pressure source 130. For example, in the case of an electrically driven pump, the voltage of an electrical circuit including the pump and/or current drawn by the pump may be monitored, and the monitored voltage and/or current may be utilized to monitor the physical characteristic of the fluid in/proximate to pressure source 130, and thus monitor the physical characteristic of the fluid in the lumen 111 of the needle 112. In an alternative embodiment, a substantial change in the voltage and/or current may be relied upon as an indication of a change in the physical characteristic of the fluid located in the pressure source 130, and hence a change in the physical characteristic of the fluid located in the lumen 111. Any device, system, method or configuration that will permit a physical characteristic of a fluid in/proximate to the pressure source 130 to be monitored, and hence permit the physical characteristic of the fluid in the lumen 111 to be monitored, may be utilized in some embodiments of the present invention.

Communication line 145 enables communication between the fluid monitor 140 and an indicator 150. Communication line 145 may be similarly constructed to communication line 135 to achieve similar performance. Indicator 150 provides, to a surgeon, medical practitioner or other user of the access catheter 100A, an indication based on the physical characteristic of the fluid in lumen 111 monitored by fluid monitor 140. In an exemplary embodiment, indicator 150 provides an indication to a user of a change of the monitored physical characteristic. By way of example and not by way of limitation, the indicator 150 may be a visual indicator such as an LED that illuminates upon a change of pressure, corresponding to a predetermined amount, of the fluid in lumen 111 of catheter needle assembly 110A. It would be appreciated that other types of indicators may be implemented in alternative embodiments of the present invention.

In an exemplary embodiment, as may be seen in FIG. 1A, the pressure source 130, the fluid monitor 140 and the indicator 150 may be combined together in a fluid control assembly contained in a housing 165. Fluid control assembly 160 may include a fluid coupling mounted on housing 165 that permits fluid supply line 120 to be easily coupled to and decoupled from the fluid control assembly 160.

FIG. 1B provides an alternate embodiment of an access catheter 100B according to embodiments of the present invention. The components of access catheter 100B are substantially the same as those of assembly 100A described above. However, in the embodiments of FIG. 1B, fluid monitor 140 utilizes a measurement taken at the catheter needle assembly 110B. In an exemplary embodiment, an electrically powered transducer that is sensitive to, for example, a pressure of a fluid and/or fluid flow rate (mass flow rate and/or volumetric flow rate), is located on the catheter needle assembly 110B. In such embodiments, the electrically operated transducer outputs an electrical signal to monitor 140, whereupon monitor 140 analyzes the electrical signal to evaluate the physical characteristic of the fluid in lumen 111.

Instead of or in addition to communication line 135, communication line 115 connects fluid monitor 145 and catheter needle assembly 110A. Communication line 115 may be similarly constructed to communication line 135 and/or communication line 145 to achieve similar performance. In an exemplary embodiment, communication line 115 is easily attachable to and detachable from catheter needle assembly 110A. In an exemplary embodiment, the communication line 115 may be co-located with fluid supply line 120 such that it extends from the fluid control assembly 160 with the fluid supply line 120 to the catheter needle assembly 110A. In an exemplary embodiment, the communication line 115 may be coupled to or otherwise embedded in the material of the fluid supply line 120. In an exemplary embodiment any device, system, method or configuration that will permit a physical characteristic of the fluid located in lumen 111 of needle 112 to be monitored may be used to practice those exemplary embodiments of the present invention.

It is noted that in an exemplary embodiment of the present invention, electrical communication line 115 may have a connector end that is compatible with monitors that are currently utilized in hospitals, and where the connector outputs a signal (electrical or otherwise) that may be read or otherwise analyzed by those monitors. By way of example only and not by way of limitation, communication line 115 may correspond, in communicative terms, to a commercially available guidewire used to obtain pressure readings inside a human heart. The electrical communication line 115 may be connected to a pressure monitor used in that hospital that would otherwise be connected to the guidewire. In an exemplary embodiment, any device, system, method or configuration that will permit the physical characteristic of the fluid to be monitored may be utilized to practice some embodiments of the present invention.

FIG. 1C is a schematic illustration of an alternate embodiment of an access catheter 100C according to an exemplary embodiment of the present invention. The components of the access catheter 100C may be substantially the same as those of the access catheter 100A and/or 100B described above. However, in the embodiments of FIG. 1C, instead of having an indicator 150 and a fluid monitor 140 that is remote from the catheter needle assembly 110A, an indicator/monitor 170 is located within catheter needle assembly 110C. The embodiment of FIG. 1C corresponds in principle to that of FIG. 1B in that the physical characteristics of a fluid in the lumen 111 of needle 112 is measured at the catheter needle assembly 110C.

Specific features of some exemplary embodiments of the present invention will now be described in greater detail.

As noted above, indicator 150 and/or indicator/monitor 170, may be a visual indicator. The indicator 150 may provide a visual indication by way of an LED, a cathode ray tube or an LCD screen, and may be in the form of a binary message (such as indicating that a monitored pressure has undergone a substantial change) and/or may indicate a value or other indicia indicative of the monitored fluid characteristic (such as the current pressure in the access catheter 100C). In the exemplary embodiment, the indicator 150 and or the indicator/monitor 170 may provide a mechanical visual indicator such as a poppet that will pop up or pop down upon a substantial change in the physical characteristic of the fluid that is monitored. In an alternative embodiment, the mechanical indicator may indicate a value or other indicia of the monitored fluid characteristic. Any visual indicator that will permit embodiments of the present invention to be practiced may be utilized in some embodiments of the present invention. More specifically, any visual indicator that will provide an indication of the physical characteristics of the fluid located in lumen 111 of needle 112 may be utilized in some embodiments of the present invention.

In other embodiments, the indicator may be an audio indicator and/or a tactile indicator. A tactile indicator may be utilized with respect to the catheter needle assembly 110C of FIG. 1C where the indicator/monitor 170 is located on/with the catheter needle assembly 110C. In an exemplary embodiment, the indicator 150 and/or the indicator/monitor 170 may include an audio indicator alone or on addition to a visual indicator. The audio indicator provides an audio indication by way of a speaker or the like, and may be in the form of a tone (beep, pulse, chime, etc.) and/or a synthesized voice, etc. The audio indicator may provide a binary message (such as indicating that a monitored pressure has undergone a substantial change) and/or may indicate a value or other indicia indicative of the monitored fluid characteristic (such as the current pressure in the access catheter 100C). Any audio indicator that will permit embodiments of the present invention to be practiced may be utilized in some embodiments of the present invention. More specifically, any audio indicator that will provide an indication of the physical characteristics of the fluid located in lumen 111 of needle 112 may be utilized in some embodiments of the present invention.

As noted above, the indicator 150 and/or the indicator/monitor 170 may provide a tactile indication of the physical characteristic of the fluid located in lumen 111 of needle 112. In an exemplary embodiment, at least with regard to the embodiment of FIG. 1C, the indicator/monitor 170 may vibrate upon a substantial change in the physical characteristic of the fluid located in lumen 111 of needle 112. In such a scenario, the user who is holding the catheter needle assembly 110C by the handle 114 will feel that vibration. Any tactile indicator that will permit embodiments of the present invention to be practiced may be utilized in some embodiments of the present invention. More specifically, any tactile indicator that will provide an indication of the physical characteristics of the fluid located in lumen 111 of needle 112 may be utilized in some embodiments of the present invention.

With respect to the fluid monitor 140 and/or the indicator/monitor 170, as detailed above, various embodiments may include monitoring various physical characteristics of a fluid. Exemplary embodiments include monitoring pressure or flow rate or other physical characteristics as disclosed herein or otherwise may be monitored at any location in the access catheter 100A, 100B and/or 100C that permits the physical characteristic of the fluid located in lumen 111 to be monitored. In an exemplary embodiment, the physical characteristic of fluid monitored by fluid monitor 140 and/or the indicator/monitor 170 may be a volume of a fluid. By way of example and not by way of limitation, the fluid in lumen 111 may be in fluid communication with a reservoir of fluid that comprises a variable volume. The volume changes upon a substantial flow of fluid and/or a substantial change of flow of fluid through lumen 111 out tip 113 of needle 112. By monitoring the variable volume, the physical characteristic of the fluid in the lumen may be monitored. With respect to the embodiment of FIG. 1C and/or the embodiment of FIG. 1B, monitor 40 and/or indicator/monitor 170 may be a component having a visually and/or an audibly variable volume such that when the volume of that component changes, an indication will be provided to a user of substantial flow of fluid and/or a substantial change of flow of fluid through lumen 111 out tip 113 of needle 112. By way of example, indicator/monitor 170 may comprise a variable volume reservoir in fluid communication with lumen 111 and may include a poppet that pops inward upon a reduction in volume of fluid in the variable volume reservoir.

As noted above, in some embodiments, monitoring a physical characteristic of the fluid located in lumen of needle includes monitoring a change in the physical characteristic of fluid located in lumen 111. By way of example and not by way of limitation, monitor 140 and/or indicator/monitor 170 may monitor for a change of a physical characteristic of the fluid corresponding to a predetermined amount within a predetermined period of time.

Exemplary methods of utilizing the access catheter 100A, 100B and 100C to explore for a space adjacent a layer of tissue will now be described, along with additional structural and systematic features of some embodiments of the present invention better described in the context of a method of using the access catheter 100A, 100B and 100C.

Initially, a user of the access catheter 100 inserts tip 113 of needle 112 into an outer layer of tissue of a patient, wherein “outer layer of tissue” is defined broadly to cover any layer of tissue located outward of a space within the patient. By way of example, the outer layer of tissue may be covered by multiple layers of tissue, such as skin or the like. The user applies a force to catheter needle assembly 110A, 110B or 110C such that tip 113 of needle is driven from an outer surface (outer side) of the outer tissue layer towards an inner surface (inner side) of the outer tissue layer.

As noted above, the fluid located in lumen 111 of needle 112 may be pressurized by pressure source 130. In an exemplary embodiment, at least once tip 113 has been inserted into the outer layer of tissue, the fluid in lumen 111 may be pressurized by pressure source 130. As noted above, if a physical restraint is located at the tip 113 of needle 112 or proximate thereto that prevents or otherwise limits fluid from flowing down the lumen 111 of needle 112 and out lumen 111 at tip 113 of needle 112. That is, the fluid in lumen 111 is in a static state, despite the increased pressure of the fluid. Conversely, in the absence of any physical restraints at the end of needle 112, the pressure applied to the fluid by pressure source 130 causes the fluid to travel down lumen 111 of needle 112 and out lumen 11 at tip 113 of needle 112. That is, the fluid is in a dynamic state.

During insertion of needle 112, into or through tissue of a patient, the tissue of the patient provides resistance to flow of the fluid out of tip 111 of needle 112. Thus, when a tip 113 of a needle 112 is located in the tissue of a patient, little to no fluid will be driven out of the lumen 111 even when the fluid is pressurized by pressure source 130. Along these lines, in an exemplary embodiment, pressure source 130 is configured to pressurize the fluid in lumen 111 to a peak level such that, when utilized with a needle 112 dimensioned and configured appropriately for an invasive medical procedure in a human such as those described herein, the tissue of the human will provide resistance to fluid flow out of tip 111 of needle 112.

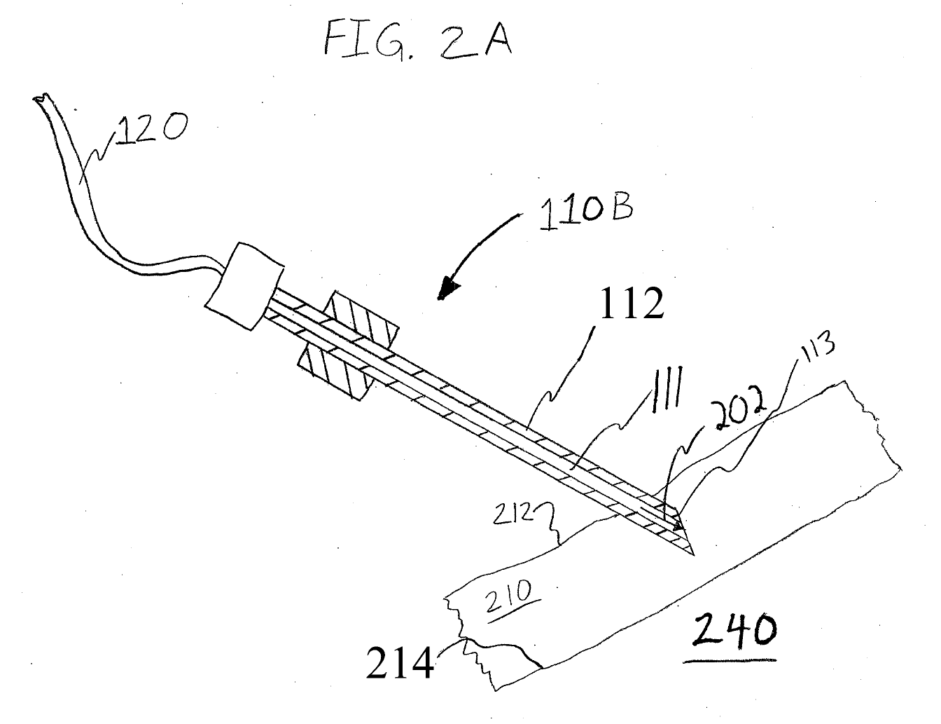

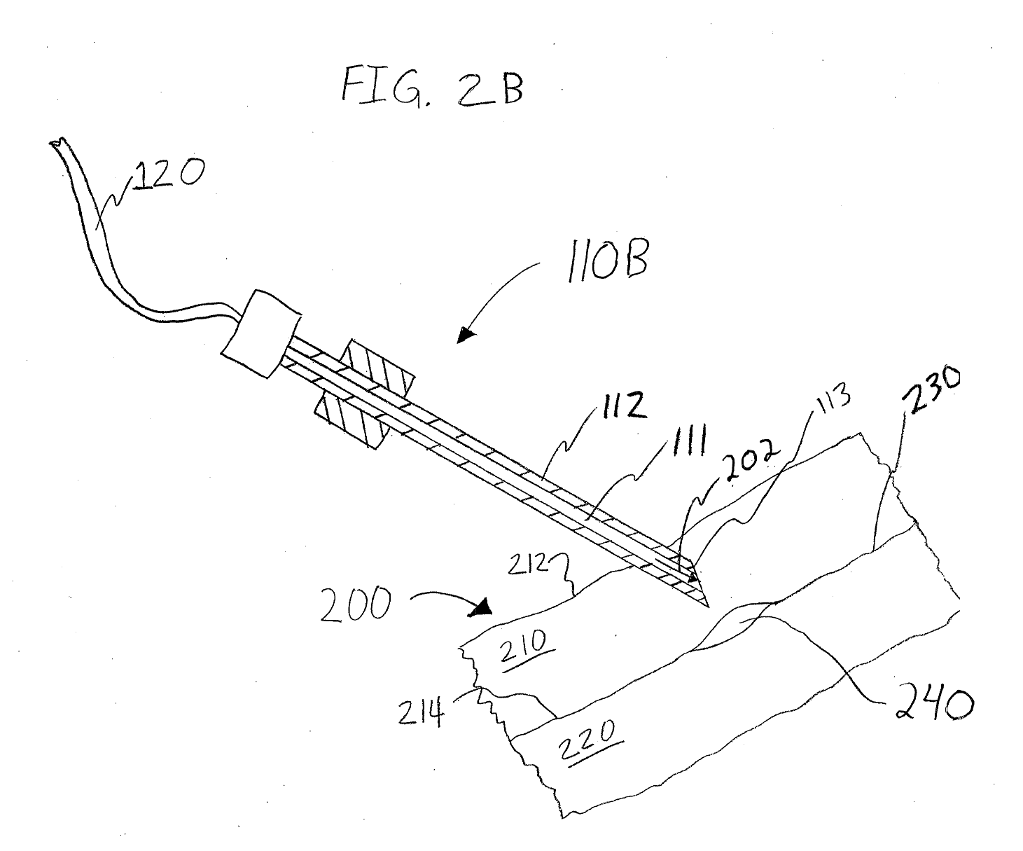

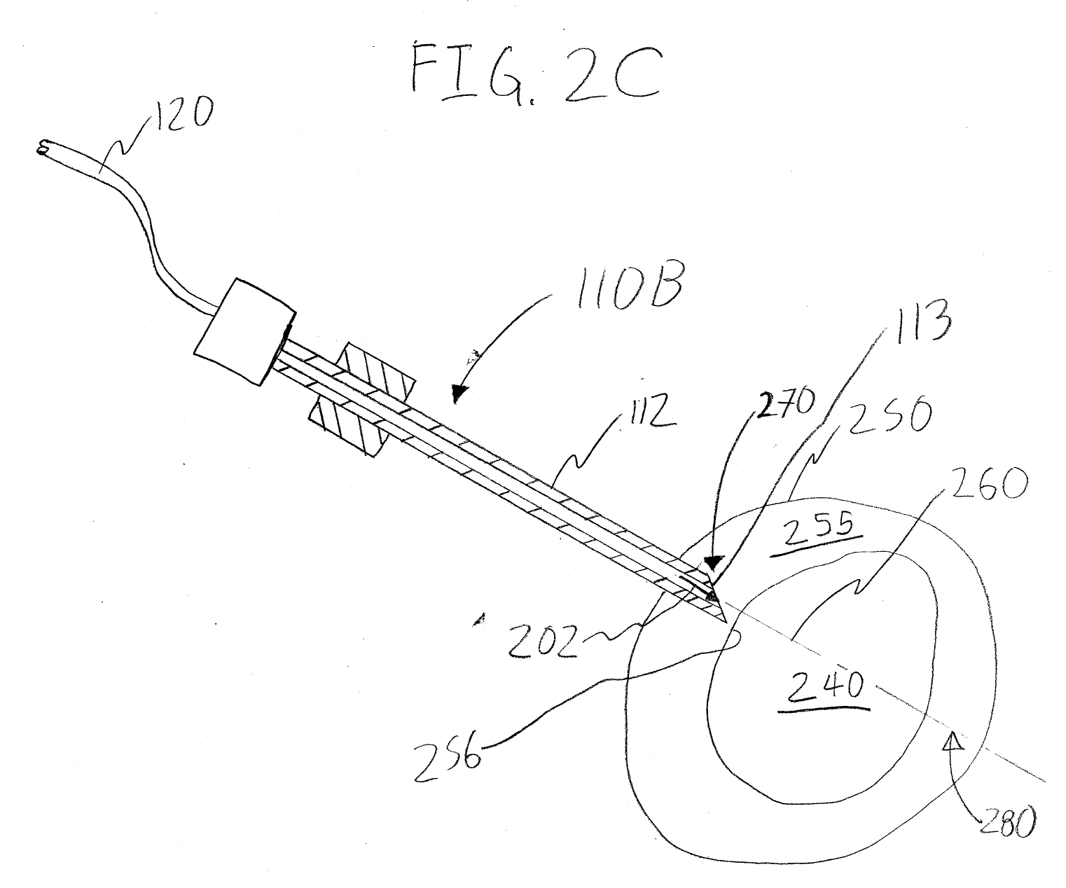

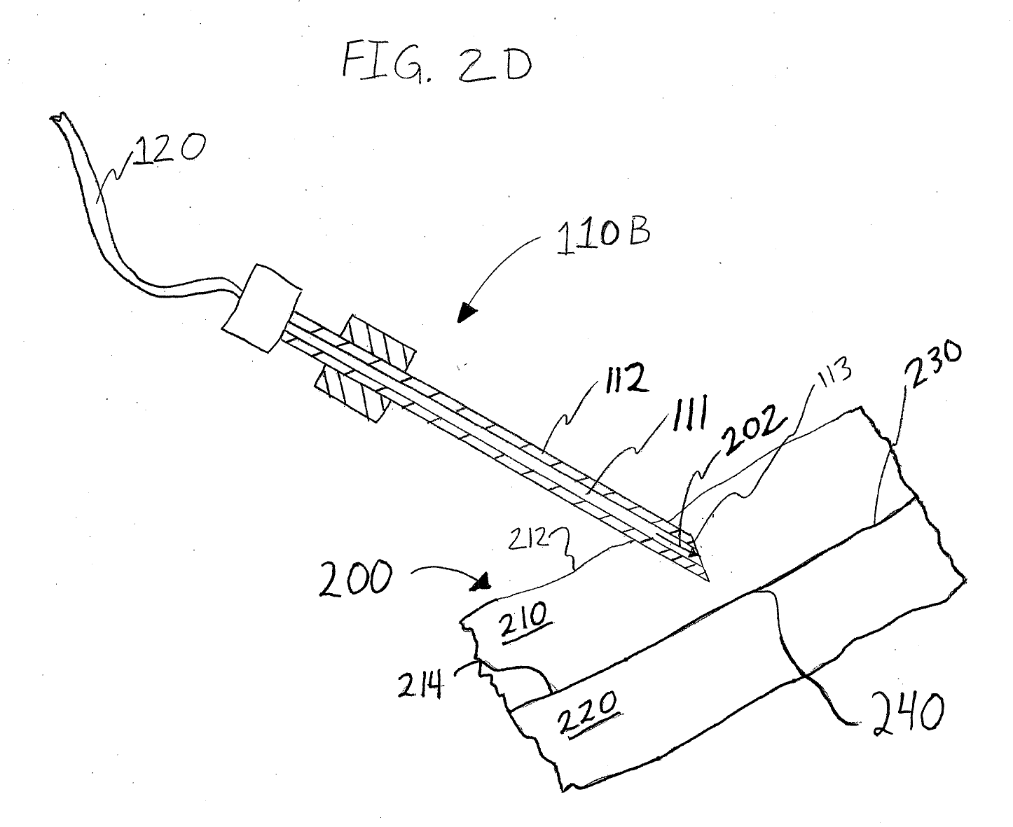

FIG. 2A is a cross-sectional view of needle assembly 110B of the embodiment of FIG. 1B. In this embodiment, communication line 115 is integral with fluid supply line 120. FIG. 2A depicts the tip 113 of needle 112 located about halfway through an outer layer of tissue 210. The layer of tissue 210 includes an outer surface 212 through which the tip 113 of the needle 112 was inserted to enter the layer of tissue 210. The layer of tissue 210 also includes an inner surface 214, as may be seen. The outer surface 212 and the inner surface 214 correspond to the outer and inner surfaces (sides) of the outer layer of tissue described above.

The tip of the needle 113 is advanced through the layer of tissue 210 until the tip 113 of the needle 112 reaches location 240 adjacent the layer of tissue 210. As may be seen in FIG. 2A, location 240 is located closer to surface 214 than surface 212 of the layer of tissue 210. Location 240 may be a space formed between two layers of tissue or the space formed in the interior of a lumen (e.g., in the case of an artery), and location 240 may further be a “potential space” formed between two layers of tissue, as will now be further detailed.

FIG. 2B depicts a first layer of tissue 210 separated from a second layer of tissue 220 of a plurality of layers of tissue 200. The two layers of tissue are separated by a plane 230. The first layer of tissue 210 and the second layer of tissue 220 respectively correspond to the outer layer of tissue and the inner layer of tissue described above. The plane 230 may be a location where the first layer of tissue 210 contacts the second layer of tissue 220 (the contact potentially being lubricated by a fluid). In FIG. 2B, location 240 corresponds to a space formed because inner surface 214 of the first layer of tissue 210 is located away or otherwise separated from the second layer of tissue 220.

Still further by way of example, in FIG. 2C, location 240 corresponds to a space formed by a lumen of, for example, an artery, vein or other vessel, etc., or the inside a cavity enclosed by tissue within a patient. Specifically, in FIG. 2C, location 240 is a space formed within artery 250 enclosed by artery wall 255.

Some aspects of FIG. 2C are the same as those of FIG. 2B in that the space at location 240 is formed by tissue surrounding the location 240 in a manner that creates a space surrounded by tissue. As with FIG. 2B, FIG. 2C depicts a space located between two layers of tissue, albeit in FIG. 2B, the layers of tissue are formed by the same tissue. In this regard, as used herein, the phrase layer of tissue encompasses a strata of tissue bounded by opposite surfaces (e.g. surfaces 212 and 214), thus forming a layer through which tip 113 of needle 112 may pierce. Thus, the same tissue folded upon itself would correspond to multiple layers of tissue. A void in tissue would also correspond to multiple layers of tissue. Thus, with respect to FIG. 2C, multiple layers of tissue are depicted, these layers lying normal to the direction of the longitudinal axis 260 of the needle 112. Specifically, artery 250 includes a first layer of tissue at position 270 in which the tip 113 of needle 112 is located, and a second layer of tissue bisected by the longitudinal axis 260 of the needle 112 at position 280 as shown in FIG. 2C, into which the tip 113 of needle 112 may enter after passing through the opposite surfaces at location 270 and passing through location 240.

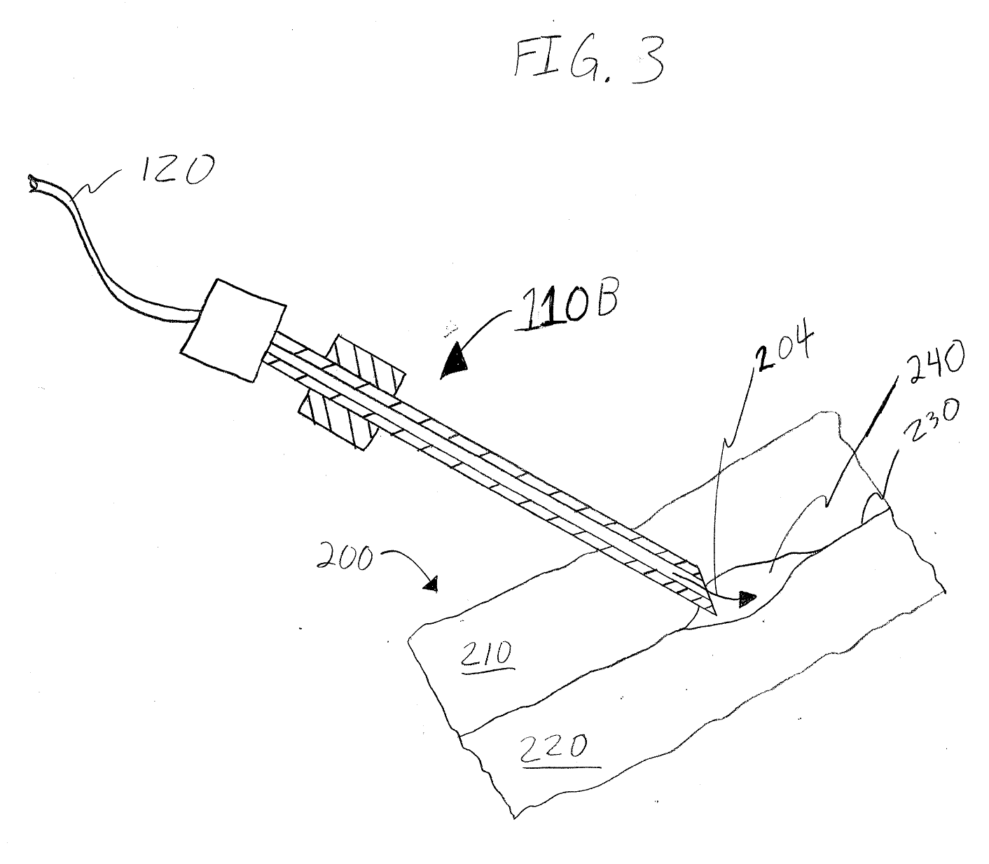

As noted above, location 240 may be a potential space. Referring to FIG. 2D, plane 230 corresponds to a potential space between the layers of tissue, including location 240 into which fluid from lumen 111 may flow as a result of the pressurization of the fluid. That is, the fluid from lumen 112 may push the layers of tissue away from each other locally about tip 113 of needle 112 as a result fluid flow from lumen 111 of needle 112, forming a space 240 as depicted in FIG. 3. With respect to FIG. 2D and FIG. 3, plane 230 corresponds to any portion of a patient between two layers of tissue that provides for substantially less resistance to fluid flow out of lumen 111 at tip 113 of needle 112 as compared to that provided by the respective tissue layers when tip 113 of needle 112 is locate in those tissue layers (i.e., tip 113 has not reached plane 230).

In the scenario depicted in FIGS. 2A-2D, the fluid in lumen 111 is pressurized, but tissue layer 210 with respect to FIGS. 2A, 2B and 2D and the tissue at location 270 with respect to FIG. 2C prevents the fluid in lumen 111 from flowing out tip 113. This phenomenon is depicted by way of example in FIGS. 2A-2D by arrow 202, which has an arrow head that stops at about the tip 113 of needle 112, as may be seen. In some instances, a tiny amount of fluid may flow from the tip 113 while the tip is surrounded by tissue, owing to the plasticity of tissue in a patient. However, this amount of fluid will be relatively minor and, in an exemplary embodiment, will not impact and/or will only slightly impact the monitored physical phenomenon of the fluid in the lumen 111. In an alternative embodiment, even if this fluid flow is not minor, an embodiment of the present invention may still monitor the physical characteristic of the fluid in lumen 111.

The user continues to apply a force to catheter needle assembly 110, continuing to drive tip 111 of needle 112 through the first layer of tissue (layer 210 with respect to FIGS. 2A, 2B and 2D and the tissue at location 270 with respect to FIG. 2C) towards the location 240, and thus towards the second layer of tissue (layer 220 with respect to FIGS. 2A, 2B and 2D and the tissue at location 280 with respect to FIG. 2C). When tip 111 of needle 112 fully penetrates the first layer of tissue (i.e., piercing inner surface 214 of FIGS. 2A, 2B and 2D, piercing inner surface 256 of artery wall 255 of FIG. 2C) and reaches location 240 adjacent to the first layer of tissue, the resistance to flow of fluid out tip 111 of a needle 112 will be substantially reduced and/or eliminated, at least relative to resistance to flow of fluid out tip 111 prior to reaching location 240 (e.g., such as the resistance to fluid flow when tip 113 of needle 112 is located as depicted in FIG. 2). Thus, a substantial amount of fluid (relative to the amount flowing out when tip 113 was obstructed, as is the case in the scenarios depicted in FIGS. 2A-2D) will flow out of lumen 111 through tip 113 of needle 112, and into location 240 (which may be a space or a potential space, where with respect to the latter, the fluid will form a space between the first layer of tissue and the second layer of tissue), as is represented by way of example in FIG. 3 by arrow 204, with the head of arrow 204. It is noted that in an exemplary embodiment where pressure source 130 provides pulsating pressurization or otherwise does not apply pressurization in a constant manner (e.g., pressure source 130 stops applying pressure or otherwise reduces the applied pressure upon a detection of a change in the monitored physical property of the fluid in lumen 111), and/or alternately provides a negative pressure to fluid in lumen 111, at least some of that substantial amount of fluid flowing out lumen 111 may be forced back into lumen 111, either as a result of plastic tendencies of tissue imparting at least a partial collapsing force on the space at location 240, and/or as a result of the vacuum effect of the negative pressure applied to the fluid in lumen 111.

In an exemplary embodiment, the flow of substantial amounts of fluid out of the tip 113 of needle 112 and/or the fact that tip 113 of needle 112 has reached location 240 may cause a change in the monitored physical phenomenon of the fluid in lumen 111. By way of example only, this may cause the pressure of the fluid located in lumen 111 of needle 112 to decrease relative to the pressure of that fluid prior to tip 113 of needle 112 reaching location 240. The new pressure and/or the pressure drop may be detected by fluid monitor 140 and/or indicator/monitor 170, depending on the embodiment utilized during the invasive procedure, and an indication will be provided to the user of this new pressure and/or the change in pressure. Based on this indication, the user may surmise that tip 113 of needle 112 is presently at location 240, which is adjacent the first layer of tissue 210 (with respect to FIGS. 2A, 2B and 2D)/the layer of tissue at location 270 (with respect to FIG. 2C). Again, as noted above, in other embodiments, the monitored physical property of the fluid in lumen 111 may be flow rate, or any other physical property that will permit a determination to be made that fluid is flowing out of lumen 111 at tip 113 of needle 112.

The method further includes stopping further insertion of tip 113 of needle 112 towards/into location 240 upon the user surmising that tip 113 of needle 112 is at location 240. In embodiments where a second layer of tissue is aligned with the longitudinal axis 260 of needle 112, as shown in FIGS. 2A-2D, the monitored physical characteristic of the fluid may be used to protect the second layer of tissue from intrusion by tip 113 and/or protecting organs beyond the second layer of tissue from intrusion by tip 113.

It is noted that in an exemplary embodiment, the fluid in lumen 111 of needle 112 may be contained therein via capillary action if the pressurization of the fluid is removed or otherwise not present. This may have utility prior to inserting tip 113 of needle 112 into an outer layer of tissue. That is, the needle 112 may be charged with fluid prior to inserting tip 113 into the tissue, thus eliminating and/or substantially reducing air contained in lumen 111, while permitting the fluid in lumen 111 to be held substantially in place prior to pressurizing that fluid.

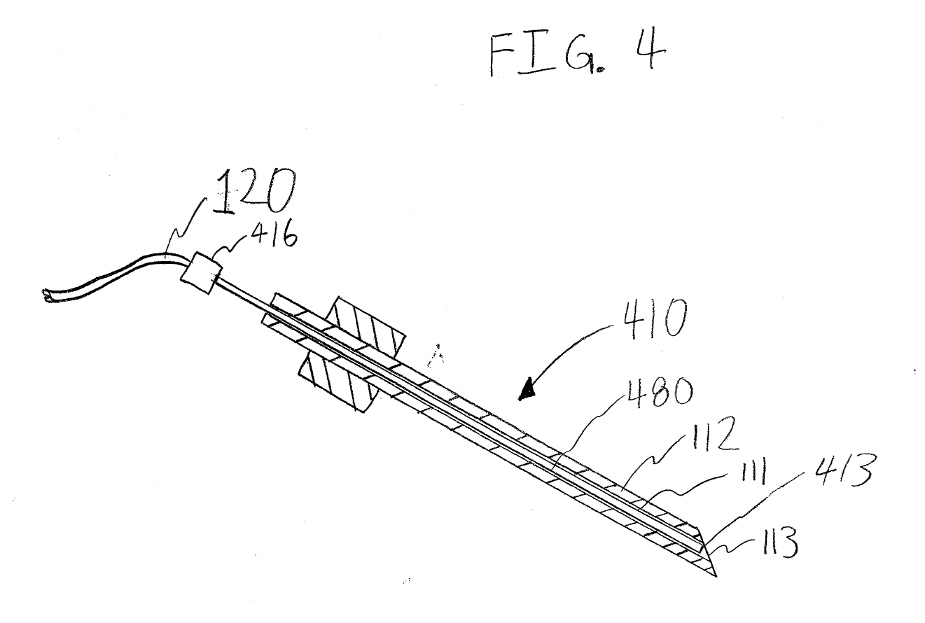

FIG. 4 depicts an alternate embodiment of the catheter needle assembly 410, wherein an interior catheter 480 is slidingly located in lumen 111 of needle 113. Embodiments of the catheter needle assembly 410 variously correspond to the catheter needle assemblies of FIGS. 1A-1C detailed above, except that in some embodiments, instead of a fluid coupling 116 in fluid communication with lumen 111 of needle 112, a fluid coupling 416 is attached to interior catheter 480, permitting fluid supply line 120 to be fluidically coupled to interior catheter 480. According to the embodiment of FIG. 4, fluid located in lumen 111 of needle 112 is also located in interior catheter 480 such that the wall of interior catheter 480 is interposed between the fluid and the walls of needle 112 forming lumen 111.

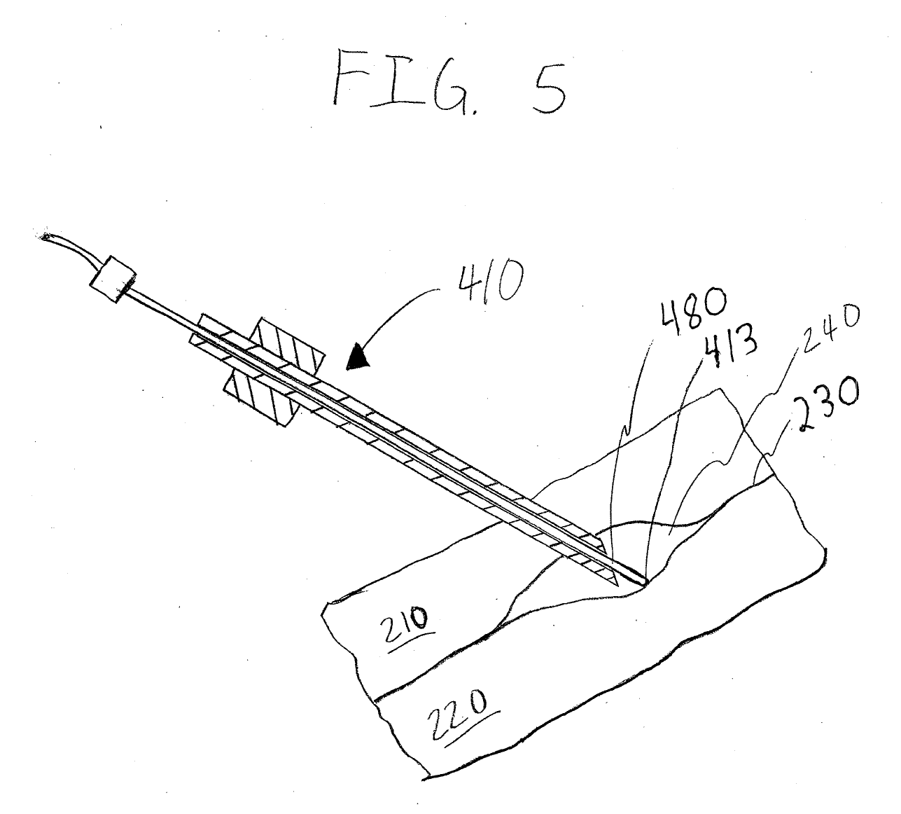

In an exemplary embodiment, the interior catheter 480 has elastic properties, or is otherwise spring loaded, and dimensioned and configured such that a tip 413 of the interior catheter 480 will automatically extend/advance outward from tip 113 of needle 112 when tip 113 of needle 112 reaches location 240, as is depicted by way of example in FIG. 5. While FIG. 5 depicts usage of this embodiment where layers 210 and 220 of separate tissue surround location 240, this embodiment may be utilized in other situations, such as that represented by FIG. 2A, FIG. 2B, FIG. 2C (e.g., where location 240 is formed by layers of the same tissue) or FIG. 2D. In an exemplary embodiment, pressurization of the fluid in interior catheter 480, and thus fluid in lumen 111 of needle 112, is achieved via the same and/or similar methods as pressurization of the fluid detailed above with respect to the embodiments of FIGS. 1A-1C. It is noted that in an exemplary embodiment, the catheter needle assembly 410 may utilize a Touhy-Borst Valve to ensure or otherwise enhance advancement of the tip 413 of interior catheter 480 from tip 113 of needle 112.

An exemplary method of utilizing the catheter needle assembly 410 of FIG. 4 includes driving tip 113 of needle 112 through, with reference to FIG. 5, the first layer of tissue 210 until tip 113 penetrates the inner surface 214 of the first layer of tissue 210. In this exemplary method, at this point, the monitored physical characteristic of the fluid in interior catheter 480, and thus lumen 111, may change in the same manner or similar manner to the changes of the physical characteristic of the fluid detailed above with respect to the embodiments depicted in FIGS. 2-3, and a user may surmise that tip 111 has reaches location 240 as detailed above, and take the actions also detailed above. Also at this point, tip 413 of interior catheter 480, which has been mostly or entirely located within lumen 111 of needle 112, springs outward as a result of, for example, the decrease in pressurization of the fluid in interior catheter 480 resulting from fluid flow out of interior catheter 480 at tip 413 upon tip 413 reaching location 240. In an alternate embodiment, tip 413 springs outward as a result of the absence of tissue applying an opposite force to tip 413 upon tip 413 reaching location 240. The method further includes continuing to push tip 113 of needle 112 towards the second layer of tissue 210 about until interior catheter tube 480, which by now is extending from tip 113 of needle 112 contacts the second layer of tissue 230, this contact providing a certain amount of resistance towards further pushing of tip 113 of needle 112 towards the second layer of tissue 220. Along these lines, the interior catheter 480 acts as a type of bumper for the catheter needle assembly 410. This resistance at least one of further providing an indication to the user that tip 113 of needle 112 is located between layers of tissue and preventing further substantial movement of tip 113 of needle 112 towards the second layer of tissue 220, thus protecting the second layer of tissue 220 from intrusion by tip 113 and/or protecting organs beyond the second layer of tissue 220 from intrusion by tip 113.

Upon driving tip 111 of needle 112 to plane 230, fluid in lumen 111 may be removed from lumen 111, by expelling the fluid out of lumen 111 at tip 113 and/or by vacuuming the fluid out of the opposite end of lumen 111. With respect to the embodiments of FIGS. 4 and 5, interior catheter 480 may be removed from lumen 111 by pulling interior catheter 480 out the opposite end of lumen 111.

When tip 111 of needle 112 is located at location 240, and, in an exemplary embodiment, after the fluid in lumen 111 is removed and/or after the interior catheter 480 is removed, the needle 112 is utilized as a catheter to accomplish various traditional catheterization procedures.

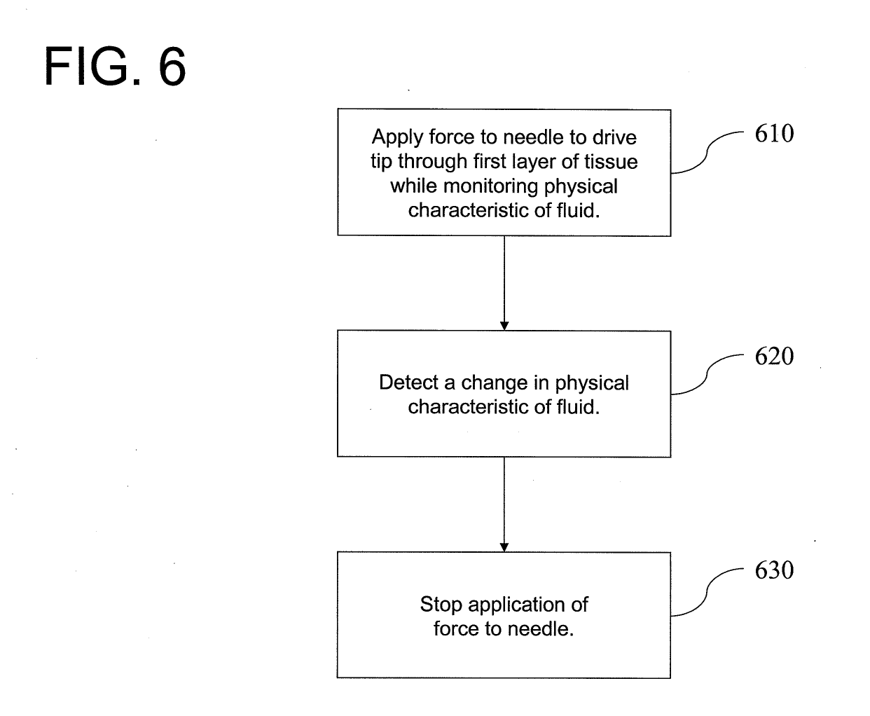

FIG. 6 provides an exemplary flow chart detailing certain steps in an exemplary invasive medical exploratory method according to some embodiments of the present invention utilizing the access catheter system according to an embodiment of the present invention. The method includes probing with needle 112 for a space and/or a potential space formed adjacent a first layer of tissue inside a patient by monitoring a physical characteristic of a fluid located in a needle lumen 111. This space and/or potential space may be formed between two layers of tissue. At step 610, force is applied to the needle 112 to drive the tip of needle through the first layer of tissue. While this force is applied, a physical characteristic of the fluid in lumen 111 of needle 112 is monitored. At step 620, a change in the physical characteristic of the fluid is detected, such as by way of example, by indicator 150 and/or indicator/monitor 170 indicating that a change has taken place. Upon detection of the change in the physical characteristic of the fluid, the application of the force onto needle 112 is halted at step 630, thus preventing tip 113 of needle 112 from further traveling in the space/potential space (and, if applicable from further traveling towards or otherwise penetrating or further penetrating into a second layer of tissue beyond the space/potential space).

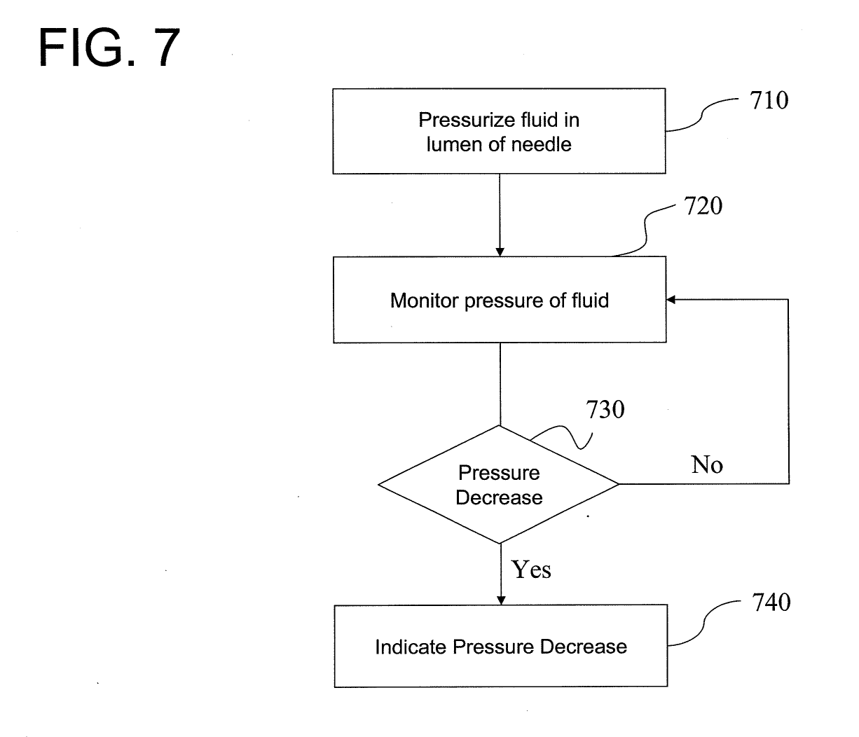

FIG. 7 depicts an exemplary flow chart detailing additional steps which may be performed as part of the method of the flow chart of FIG. 6, a portion of which may be embodied in a computer algorithm in the fluid control assembly 160 detailed above, according to an embodiment of the present invention. At least once tip 113 of needle 112 is in the first layer of tissue, or is otherwise positioned below the outermost layer of skin or the like, fluid in lumen 111 is pressurized at step 710. A physical characteristic such as pressure of the fluid in lumen 111 is automatically monitored at step 720 by any device, system, method or configuration, including hardware, software, firmware, etc., such that a change in the physical characteristic of the fluid may be detected. Specifically, at step 730, the pressure of the fluid in lumen 111 is evaluated. If no pressure decrease is detected that corresponds to predefined parameters (e.g., a decrease in pressure of a certain percent or p.s.i. optionally within a certain amount of time), or if otherwise a determination is made that the fluid in lumen 111 is in a static state, the method includes going back to step 720 and again monitoring the pressure of the fluid in the lumen 111. If a pressure decrease is detected that corresponds to predefined parameters, or if otherwise a determination is made that the fluid in lumen 111 is in a dynamic state, the method proceeds to step 740, whereupon indicator 150 and/or indicator/monitor 170 provides an indication to a user of the pressure change. In this embodiment, the pressure decrease in the fluid in the lumen 111 is a result of tip 113 of needle 112 being located a space and/or a potential space, whereupon fluid in lumen 111 flows out of the lumen 111 at tip 113. Thus, the indication of the pressure drop is indicative of tip 113 of needle 112 being located at a space and/or a potential space.

In an embodiment utilizing the method detailed with respect to FIG. 7, upon a determination that the physical characteristic of the fluid located in the interior of the lumen is indicative of fluid in a static state, which includes the absence of an indication that the fluid is indicative of fluid in a dynamic state, the user maintains insertion force on needle 112, continuing to drive tip 113 into the first layer of tissue. Alternatively, a user may follow an incremental process. This incremental process may be a look-insert-look approach, whereby the user “looks” or otherwise evaluates whether there is an indication of fluid in a dynamic state before reapplying an insertion force to the needle to continue driving tip 113 into the first layer of tissue. Upon a lack of an indication that the fluid is in a dynamic state, the user may then again apply an insertion force to drive tip 113 into the tissue a bit more, and then stop and “look’ or otherwise evaluate whether there is now an indication of fluid in a dynamic state. This process may be repeated until an indication is provided to the user that the fluid is in a dynamic state, which is thus indicative of tip 113 of needle 112 being located at the space or the potential space.

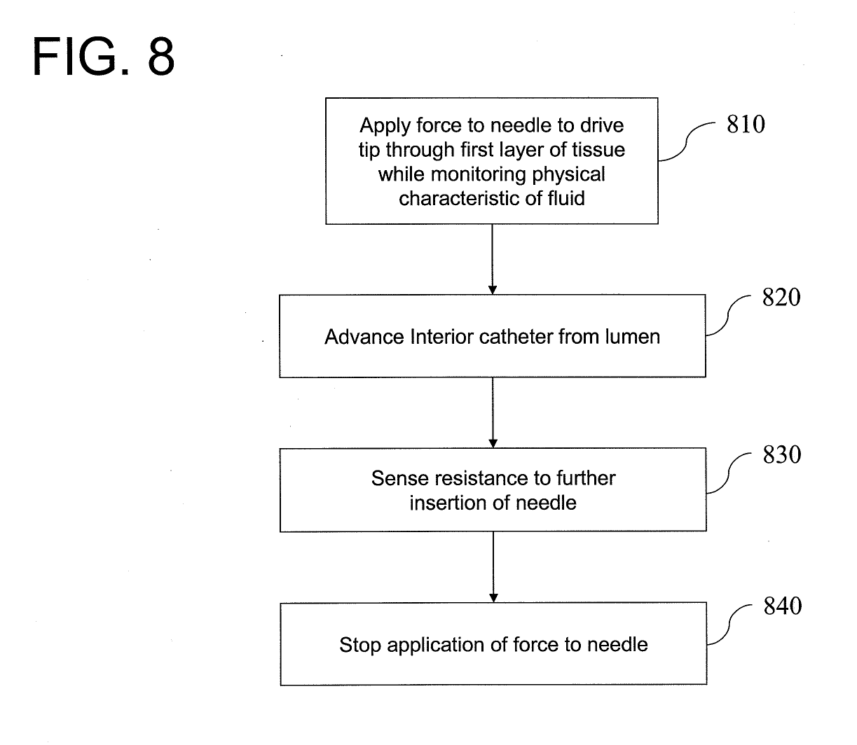

FIG. 8 provides another exemplary flow chart detailing certain steps in an exemplary invasive medical exploratory method according to some embodiments of the present invention involving probing with a needle 112 for a space or a potential space between a first layer of tissue and a second layer of tissue inside a patient, wherein the interior catheter 480 is positioned in lumen 111 of needle 112. At step 810, force is applied to the needle 112 to drive the tip of needle through the first layer of tissue. While this force is applied, a physical characteristic of the fluid in lumen 111 of needle 112 may be monitored. Also while this force is applied, at least while tip 113 of needle 112 is located in a layer of tissue, tip 413 of interior catheter 480 is at least substantially located in lumen 111. At step 820, tip 413 springs outward from tip 113 of needle 112 as a result of tip 113 reaching a space and/or a potential space. At step 830, the user who is applying the force on needle 112 to drive tip 113 through the first layer of tissue senses resistance to further insertion of needle 112 as a result of tip 413 contacting a second layer of tissue. Upon sensing the resistance to further insertion of needle 112, the application of the force onto needle 112 is halted at step 840, thus preventing tip 113 of needle 112 from further traveling towards, or otherwise penetrating or further penetrating into the second layer of tissue 220. In an embodiment, an incremental approach may be utilized with interior catheter 480 in a manner the same as or similar to the incremental approach described above.

As noted above, an exemplary invasive medical exploratory method according to some embodiments of the present invention includes driving a needle, or, more specifically, driving the tip of the needle through multiple layers of tissue to locate the tip of the needle at a space and/or a potential space adjacent to the last layer of tissue through which the tip of the needle is driven. This embodiment may be practiced, when, for example, performing a amniocentesis.

In this method, pressure is pulsatingly applied to the fluid in the needle lumen, although in other embodiments, the fluid is simply pressurized and held at a relatively constant level. The pressure is applied at least after the tip of the needle is inserted into one of the layers of tissue.

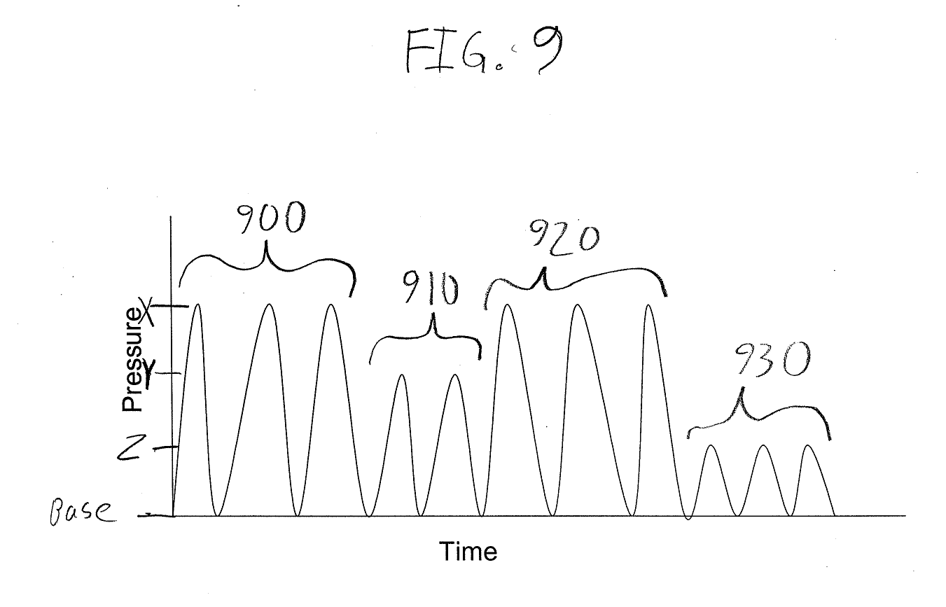

FIG. 9 depicts a graph of pressure of the fluid in the needle lumen vs. time, where time progresses as the tip of the needle is driven through the multiple layers of tissue. Specifically, during time period 900, the pressure of the fluid in the lumen oscillates between a baseline pressure (“Base”) and a pressure X, the oscillation being due to the pulsatile application of the pressure to the fluid. During time period 900, the tip of the needle is located in a first layer of tissue (i.e., the tip has not yet fully penetrated that layer of tissue) and is being advanced through the first layer of tissue. During time period 900, the user is monitoring the pressure of the fluid in the lumen. At the end of time period 900, the tip of the needle penetrates the first layer of tissue, and enters the location (a space and/or a potential space) between the first layer and a second layer. Hence, the pressure of the fluid oscillates between the baseline pressure and a pressure Y, pressure Y being lower than pressure X because more of the fluid flows out the lumen of the needle than when the tip of the needle was in the first layer of tissue. The user, who is monitoring the pressure of the fluid within the lumen identifies this pressure change. However, because the user of the system understands that multiple layers of tissue must be penetrated before reaching the desired location, the user continues to advance the tip of the needle during time period 910, such that at the end of time period 910, the tip of the needle enters the second layer of tissue. During time period 920, the tip of the needle is advanced through the second layer, and the pressure oscillates as depicted. The user continues to monitor the pressure of the fluid.

At the end of time period 920, the tip of the needle penetrates the second layer of tissue, and enters a location (a space and/or a potential space) adjacent the second layer opposite the first layer. Hence, during time period 930, the pressure of the fluid oscillates between the baseline pressure and a pressure Z, pressure Z being lower than pressure X because more of the fluid flows out the lumen of the needle than when the tip of the needle was in the second layer of tissue. The user, who is monitoring the pressure of the fluid within the lumen identifies this pressure change. Because the user knows the number of layers of tissue that must be penetrated to reach the desired location (space and/or potential space), the user knows that this “second” pressure drop (the pressure drop at time period 930) is indicative of the tip of the needle being located at the desired location, and the user ceases advancing the tip of the needle.

It is noted that while FIG. 9 presents two pressure drops, more pressure drops may be experienced depending on the number of layers of tissue through which the tip of the needle penetrates while the pressure of the fluid is being monitored.

Still further, in an exemplary embodiment, localized characteristics of the pressure drop may be evaluated to determine where the tip of the needle is located. For example, FIG. 9 depicts the maximum pressure during time period 930 as being different than the maximum pressure during time periods 900, 910 and 920. The maximum pressure during time period 930 may be known to correspond to the maximum pressure that would exist when the tip of the needle reaches a desired location, and thus the user would know, irrespective of the number of pressure drops, that the tip of the needle is located at the desired location.

It is noted that while the graph of FIG. 9 depicts pressure change vs. time, the graph could also represent other changes in the physical characteristic of the fluid within the lumen, such as, for example, flow rate, etc., as detailed herein.

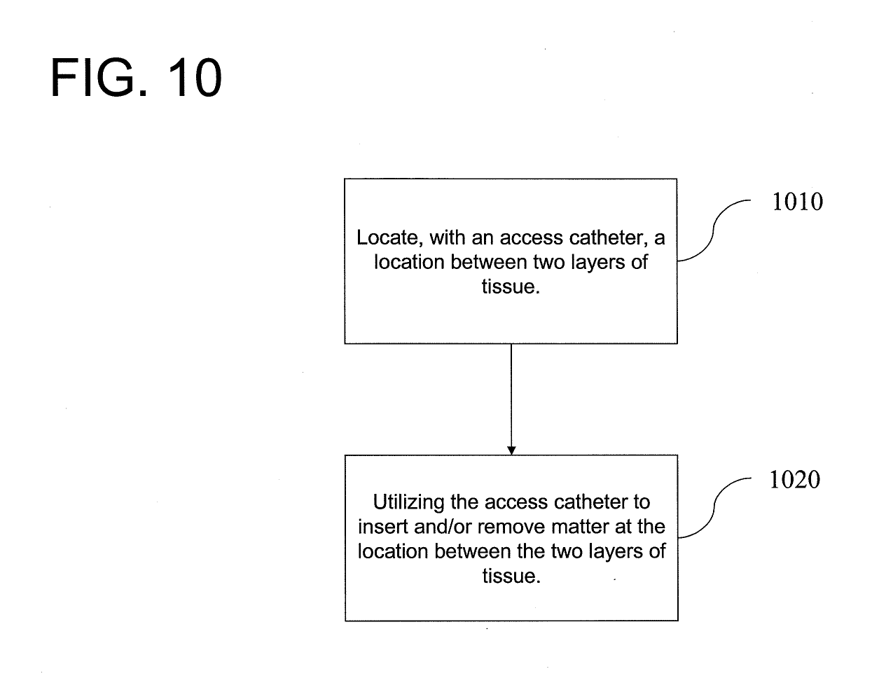

FIG. 10 presents an exemplary flow chart utilizing the access catheter. At step 1010, the access catheter is utilized to locate a space adjacent a layer of tissue. In an exemplary embodiment, step 1010 may be executed using some or all of the methods disclosed herein, and variations thereof. After locating the space adjacent the layer of tissue, the access catheter utilized to locate the space is used at step 920 to insert and/or remove material (e.g., unwanted fluid located between the two layers, an electrical tissue stimulator, a monitor component, etc.) at the space. This action being consistent with the above-mentioned uses of needle 112 once tip 113 is located at location 240.

In some embodiments, the access catheter system may be used to determine whether the tip of the needle is inside of an artery or a vein. In this regard, if the tip of the needle is located in an artery, pressure of the fluid in the lumen of the needle will be substantially higher than what it would be if the tip of the needle is located in a vein. This is because the pressure of the blood in an artery of a patient is higher than the pressure of blood in a vein of the patient (on the order of between two and four times higher, and thus the pressure of the fluid in the lumen may correspondingly be two to for times higher in the case of an artery vs. a vein). Accordingly, a method of using the access catheter system as disclosed herein may include inserting a tip of a needle into the space on the inside of what is believed to be an artery or a vein. The method may further include indicating to a user the pressure of the fluid in the lumen once the tip of the needle is on the inside of the artery or vein. The user may evaluate the pressure of the fluid within the lumen, and determine whether the tip of the needle is in an artery or a vein based on the pressure (a higher relative pressure meaning that the tip of the needle is in an artery, a lower relative pressure meaning that the tip of the needle is in a vein).

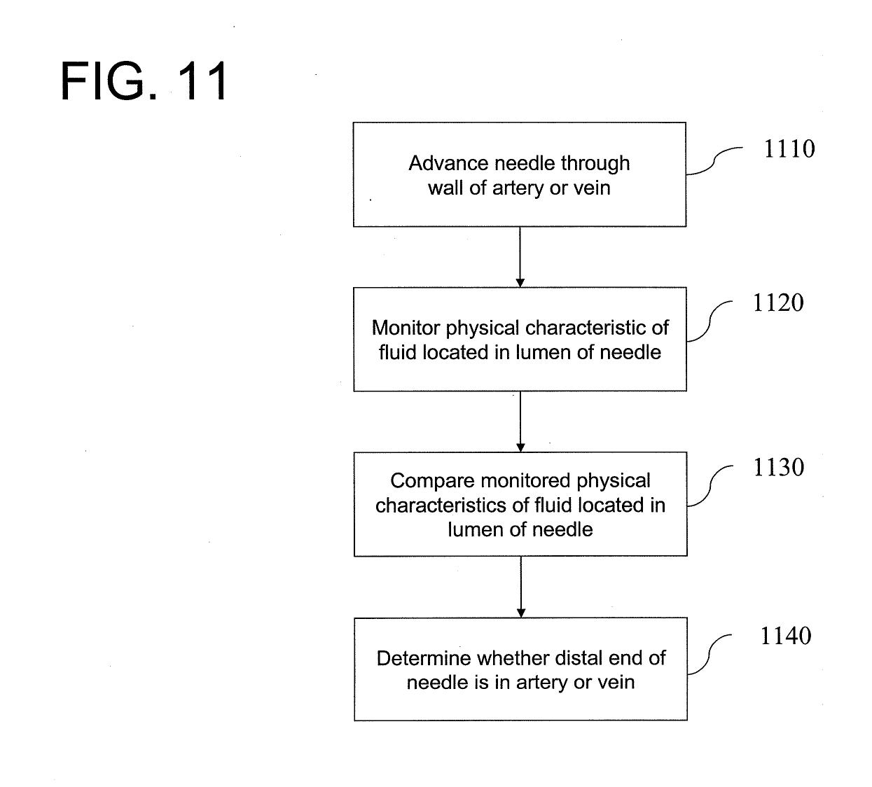

In view of the above, FIG. 11 presents an exemplary flow chart of an invasive medical method to determine whether a distal end of a needle having a fluid-filled lumen is located in the lumen of an artery or the lumen of the vein of a patient. At step 1110, the needle is advanced through a wall of an artery or a vein until the distal end of the needle is in the lumen of the artery or the lumen of the vein. At step 1120, a physical characteristic of the fluid located in the needle lumen is monitored when the distal end of the needle is in the lumen of the artery or the vein. At step 1130, the monitored physical characteristic of the fluid located in the needle lumen is compared to at least one of a known fluid characteristic indicative of the distal end of the needle being located in a lumen of an artery or a known fluid characteristic indicative of the distal end of the needle being located in a lumen of a vein. These known fluid characteristics may be based on statistical samples of a statistically substantial number of patients and/or may be directly based on the patient into which the needle has been inserted. Step 1130 may be executed automatically by the system, where the known fluid characteristic is stored in a look-up table or the like in a memory that can be accessed by a computer.

At step 1140, a determination is made based on the comparison made at step 1130 as to whether the distal end of the lumen is located in at least one of a lumen of an artery or a lumen of a vein. In an exemplary embodiment, if a determination is made at step 1140 that the distal end of the needle is in the lumen of a vein or not in the lumen of an artery, the method further includes withdrawing the tip of the needle from the lumen of the vein or other organ, and inserting the tip of the needle into an artery or another vein and repeating steps 1120-1140 again (or visa-versa if it is desired to position the tip of the needle in a lumen of a vein).

Embodiments of the present invention have been described with reference to surgical procedures performed on a patient. It would be appreciated that the term patient has been used merely for ease of illustration, and should not be construed to limit embodiments or use of the present invention. For example, the device and method of the present invention may be used in any context in which it is desirable to precisely position the distal end of a needle. For example, embodiments of the present invention may be used in living and deceases human and non-human organisms, or organs, tissue, etc., extracted from a organism.

The invention described and claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

What is claimed is:

1. An access catheter system, comprising:
a needle assembly comprising a needle having an elongate lumen configured to be substantially filled with a fluid;
a fluid monitor configured to monitor a physical characteristic of fluid within the lumen, and configured to sense a change in the physical characteristic of the fluid; and
an indicator configured to provide a indication of the sensed change in the physical characteristic of the fluid.

2. The access catheter system of claim 1, further comprising:
a pressure source configured to pressurize the fluid in the lumen.

3. The access catheter of claim 2, wherein the pressure source is configured to apply pulsatile pressure to the fluid in the lumen.

4. The access catheter of claim 1, wherein the needle assembly comprises:
an interior catheter slidably positioned in the lumen, wherein the interior catheter is configured to extend from the distal end of the needle lumen, and wherein the interior catheter tube is fluidically coupled to a pump positioned outside the need assembly.

5. The access catheter of claim 4, wherein the interior catheter is formed from an elastomer material, and wherein the interior catheter is configured to elastically compress such that at least a substantial portion of the portion of the interior catheter extending outward of the needle is compressed inwardly into the needle lumen in response to contact with tissue.

6. The access catheter of claim 2, wherein the pressure source is fluidically coupled to the lumen in the needle via fluid supply line, and wherein the fluid monitor is configured to monitor the physical characteristic of the fluid in the lumen by monitoring a characteristic of the fluid in at least one of the pressure source and the supply line.

7. The access catheter of claim 2, wherein the pressure source is fluidically coupled to the lumen in the needle via fluid supply line, and wherein the monitor is configured to sense a change in at least one of the voltage of an electrical circuit including the pressure source and the current drawn by the pressure source.

8. The access catheter of claim 1, wherein the monitor is a flow sensor and is configured to monitor the at least one of the flow rate of the fluid into the proximal end of the lumen and the flow rate of the fluid out of the distal end of the lumen.

9. The access catheter of claim 1, wherein the fluid monitor and indicator are positioned in a housing that is physically separate from the needle assembly.

10. The access catheter of claim 1, wherein the fluid monitor and indicator are positioned in the needle assembly.

11. The access catheter of claim 1, wherein the monitor is a pressure sensor and is configured to monitor the pressure of the fluid in the lumen.

12. The access catheter of claim 1, wherein the monitor is a fluid volume sensor and is configured to monitor changes in the volume of the fluid in the lumen.

13. The access catheter of claim 1, wherein the indicator is a visual indicator.

14. The access catheter of claim 1, wherein the indicator is an audible indicator.

15. The access catheter of claim 1, wherein the indicator is a tactile indicator.

16. An operational method of an access catheter including a needle assembly comprising a needle having an elongate lumen configured to be substantially filled with a fluid, the method comprising:
monitoring a physical characteristic of fluid located in the needle lumen while the needle is advanced in a patient;
sensing a change in the monitored physical characteristic; and
providing an indication of the sensed change.

17. The method of claim 16, further comprising:
pressurizing the fluid in the lumen.

18. The method of claim 17, further comprising:
applying pulsitative pressure to the fluid in the lumen.

19. The method of claim 17, wherein sensing a change in the monitored physical characteristic of the fluid comprises:
sensing a decrease of pressurization of the fluid located in the needle lumen.

20. The method of claim 16, wherein sensing a change in the monitored physical characteristic of the fluid comprises:
sensing a change in at least one of the flow rate of the fluid into the proximal end of the lumen and the flow rate of the fluid out of the distal end of the lumen.

21. The method of claim 16, wherein sensing a change in the monitored physical characteristic of the fluid comprises:
sensing a change in the volume of the fluid in the lumen.

22. The method of claim 16, wherein the needle assembly comprises an interior catheter slidably positioned in the lumen, and wherein the method further comprises:
advancing the interior catheter from a distal end of the needle lumen.

23. The method of claim 16, wherein providing an indication of the sensed change comprises:
providing a visual indication of the sensed change.

24. The method of claim 16, wherein providing an indication of the sensed change comprises:
providing an audible indication of the sensed change.

25. The method of claim 16, wherein providing an indication of the sensed change comprises:
providing a tactile indication of the sensed change.

26. A method to position a distal end of a needle having a fluid-filled lumen at a space and/or a potential space inside a mammal adjacent a first side of a first layer of tissue of the mammal, comprising:
inserting the needle into the first layer of tissue through a second side of the first layer of tissue opposite the first side;
advancing the needle through the first layer of tissue in a direction from the second side towards the first side;
monitoring a physical characteristic of the fluid located in the needle lumen while advancing the needle; and
ceasing advancement of the needle when the monitored physical characteristic of the fluid located in the needle lumen indicates that the distal end of the needle has entered the space and/or the potential space.

27. The method of claim 26, wherein monitoring the physical characteristic of the fluid located in the needle lumen includes identifying a decrease of pressurization of the fluid located in the needle lumen.

28. The method of claim 26, further comprising:
pressurizing the fluid located in the needle lumen,
wherein monitoring the physical characteristic of the fluid located in the needle lumen includes identifying a decrease of pressurization of the fluid located in the needle lumen.

29. The method of claim 26, comprising repeatedly:
increasing pressure of the fluid in the needle lumen to a first pressure greater than a second pressure; and
permitting the pressure of the fluid in the needle lumen to decrease from the first pressure towards the second pressure.

30. The method of claim 26, wherein the fluid is a saline solution, the method further comprising:
applying micro-water hammer to at least the first layer of tissue using the saline solution while advancing the needle through the first layer of tissue.

31. The method of claim 26, wherein:
monitoring the physical characteristic of the fluid includes identifying a change in the physical characteristic of the fluid located in the lumen that is indicative of fluid flow through the lumen resulting from the distal end of a needle being located in the space and/or the potential space.

32. The method of claim 26, wherein the space and/or the potential space is formed between a first layer of tissue and a second layer of tissue, the second layer of tissue being closer to the first side than the second side of the first layer of tissue, wherein the method includes:
pushing the distal end of the needle through the first layer and pushing the distal end of the needle towards the second layer of tissue only until the monitored physical characteristic of the fluid in the needle lumen is indicative of fluid flowing through the lumen

33. The method of claim 26, wherein the method includes:
pushing the distal end of the needle through the first layer only until the monitored physical characteristic of the fluid in the needle lumen is indicative of at least one of:
the distal end of the needle having completely penetrated first layer of tissue; and
the distal end of the needle being located between the first layer of tissue and a second layer of tissue, wherein the second layer of tissue is closer to the first side than the second side of the first layer of tissue.

34. The method of claim 26, wherein the physical characteristic is at least one of pressure or pressure change of the fluid in the lumen.

35. The method of claim 26, wherein the method includes:
pushing the distal end of the needle through the first layer; and
pushing the distal end of the needle towards the second layer only until an interior catheter tube extending from at least a tip of the needle through the lumen contacts the second layer.

36. The method of claim 26, wherein the method includes:
applying a pushing force to the needle to push the needle through the first layer of tissue; and
pushing the distal end of the needle into the space only until an interior catheter tube extending through the lumen from the distal end of the needle provides noticeable resistance to the pushing force.

37. The method of claim 26, wherein an interior catheter tube is located in the needle lumen, wherein the method includes:
applying a pushing force to the needle to push the distal end of the needle through the first layer and into the space;
after pushing the distal end of the needle into the space, automatically springingly extending from the distal end of the needle a portion of the interior catheter tube; and
pushing the tip of the needle towards a second layer of tissue until a portion of the interior catheter tube extending from the distal end of the needle at least one of contacts the second layer of tissue or provides resistance to the pushing force as a result of contact with the second layer of tissue,
wherein the second layer of tissue is closer to the first side than the second side of the first layer of tissue.
